(19) United States
(12) Patent Application Publication
(10) Pub. No.: US 2024/0115871 A1
(43) Pub. Date: Apr. 11, 2024

(54) MACHINE AND METHOD FOR TREATING VISCERAL FAT

(71) Applicant: Maria del Pilar SANCHEZ JAIME, SANT JOAN DESPI (ES)

(72) Inventor: Maria del Pilar SANCHEZ JAIME, SANT JOAN DESPI (ES)

(21) Appl. No.: 18/225,157

(22) Filed: Jul. 23, 2023

(30) Foreign Application Priority Data
Oct. 6, 2022 (ES) P202230865

(51) Int. Cl. A61N 1/40

(52) U.S. Cl. CPC A61N 1/403

(57) ABSTRACT

A machine for therapeutic treatment to reduce visceral fat in a patient has four channels each linked to electrodes for the application of electromagnetic currents to be applied to a specific region of a patient, managed by a specific software program that provides a power value for each of the channels in an asynchronous way, such that it allows for the simultaneous readjustment of the power value of each of the four channels, depending on at least the energy flow value emitted by the active electrodes of each channel and the energy flow value received from each of the passive electrodes of the corresponding channels.

### CROSS-REFERENCE TO RELATED APPLICATION

This application claims priority to Spanish Patent Application P202230865 filed on Oct. 6, 2022, which is hereby incorporated by reference for all purposes in its entirety.

### FIELD OF THE INVENTION

The field of the invention is directed to manufacturing equipment, apparatuses and devices for therapeutic medical treatments, focused particularly on those that apply electric current.

### BACKGROUND OF THE INVENTION

Nowadays, therapeutic machines and devices that apply currents or high frequency electromagnetic fields to the bones, muscles or tissues of the human body which use current-generating machines linked to one or more electrodes mounted on a specific accessory for the area to be treated are well known.

Although these devices have been proven to be effective in different therapeutic treatments through the application of electric currents, they are limited when it comes to applying treatments aimed at fat located in deep areas of the human body and which may imply a risk for a patient, such as visceral fat.

It is worth mentioning that visceral fat is an important aspect, given that it can be harmful to human health and a precursor of metabolic and cardiovascular diseases, and even some types of cancer.

As such, visceral fat is a public health problem that uses the metabolic pathway to cause several widespread pathologies. Excess visceral fat causes insulin resistance, metabolic syndrome, is a catalyst for the development of different types of cancers and accelerates aging. Currently, 52% of the world's population is overweight or obese, 30% suffer from hypertension and 9% are diabetic, and therefore it is necessary to reduce visceral fat to improve all of these conditions.

Current methods for helping obese people lose visceral fat are mainly focused on physical exercise and depend on the intensity, frequency and characteristics of the subject with heart rates higher than 160 beats/min. and an oxygen consumption from 55% to over 70%.

Although radiofrequency devices that are generally applicable for treating skin, muscular or bone conditions, along with other ailments, in addition to aesthetic treatments, are known, the applicant is unaware of a radiofrequency-based device capable of reducing the volume of visceral fat.

Furthermore, the applicant is currently unaware of the existence of an invention having all the features described herein.

### OBJECT OF THE INVENTION

The object of this application is to register a machine for treating visceral fat, as well as a method for treating the same.

More specifically, the invention proposes the development of a machine for therapeutic treatment related to visceral fat, provided with four channels each linked to electrodes for electromagnetic currents to be applied to a specific region of a patient, managed by a specific software program that allows for a synchronised operation by means of a single power source.

### DESCRIPTION OF THE INVENTION

The present invention has been developed for the purpose of providing a therapeutic molecular electromagnetic resonance or diathermy machine configured as a novelty in the field of the application and solves the aforementioned drawbacks, also providing other additional advantages that will be evident from the accompanying description below.

It is therefore an object of the present invention to provide a machine for the application of therapeutic treatments in which the visceral fat of a patient is involved, of the type comprising a current-generating machine, first patient interface means, a computerised control module connected to the patient interface, said computerised control module being capable of automating treatments according to a time and temperature pattern after the necessary parameters are entered. In particular, the invention is characterised in that it comprises four channels, each one of them provided with a radiofrequency generator connected to active electrodes and passive electrodes, a temperature control circuit connected to temperature sensors arranged in the monopolar active electrodes that allow the temperature value in the body area that is in contact with the electrodes to be known, and a specific software program housed in the computerised control module that provides a power value for each of the channels in an asynchronous way, such that it simultaneously readjusts the power value of each of the four channels depending on the energy value emitted by the active electrodes of each channel and the energy value received from each one of the passive electrodes of the corresponding channels, as well as other parameters, such as the frequency value, the temperature and the thickness of the lipid layer.

Thanks to these features, it is possible to supply the energy required to work in depth on a patient's visceral fat through the arrangement of four channels that can work simultaneously and asynchronously, without producing undesirable side effects on the patient's skin or causing visceral inflammation.

Thus, the machine allows energy to be concentrated in a focused manner on a specific area of a patient's body, such as the abdomen, being able to safely liquefy the fat located around the viscera.

Preferably, the first interface means comprise a touchscreen, such that it does not require a keyboard with mechanical buttons, although it is also possible to use a mechanical keyboard instead of a touchscreen.

Additionally, the machine can include a second brain-machine patient interface, providing the user/patient with a safety control over the temperature of the device, which has an EEG capture terminal, with a headband-shaped fastening element, configured to detect brain impulses of the patient or operator, the terminal being connected to the computerised control module.

The described machine for therapeutic treatment therefore represents an innovative structure with structural and constitutional features hitherto unknown for the intended purpose thereof, reasons which, taken together with its usefulness, provide it with sufficient grounds for obtaining the requested exclusivity privilege.

The active and/or passive electrodes of the present invention may preferably be electrodes applied to the skin.

In one embodiment, the active and/or passive electrodes may be grouped on a strap that is configured to be placed on the surface of the patient's body, thus simplifying the placement of the electrodes on the patient.

According to the invention, the computerised control module can execute the software program to automatically regulate and/or stop the flow of energy transferred through at least one of the channels with the aim of performing, or optimising, the therapy, depending on the different body parameters of the patient being treated.

Another object of the invention provides a method for treating visceral fat, which uses a machine as described above, which comprises the application of an energy flow by means of the arrangement of a plurality of active electrodes on the patient's skin in such a way that the input energy flow is produced in a focused way (meaning, the energy is directed so that it is concentrated in a specific region inside the patient), each electrode being active, and passive electrodes (envisaged for the output current) linked to a corresponding channel of the machine,

By means of a software program each of the channels has the ability to share data with each other, and apply an energy flow based on the ratio of the input energy flow value to the output energy flow value. It must be mentioned that this adaptation depends on the patient's body fat that is found between the antennae that are present on the active and passive electrodes.

According to another aspect of the invention, the software program is executed to apply an energy flow with a specific temperature value based on the ratio of the input energy flow value to the output energy flow value.

Preferably, the active and passive electrodes have a radial arrangement around the patient, such that the active electrodes face the passive electrodes associated with one same energy flow channel, giving rise to energy interference in the central part of a patient's body, generating an amplified and concentrated signal pattern.

Other features and advantages of the machine for therapeutic treatment object of the present invention will be apparent from the description of a preferred, but not exclusive, embodiment, which is illustrated by way of non-limiting example in the accompanying drawings, in which:

### BRIEF DESCRIPTION OF THE DRAWINGS

FIG. 1 is a schematic isometric view of an embodiment of the machine for therapeutic treatment of visceral fat according to the present invention;

FIG. 2 is a schematic view of the machine according to the invention;

FIG. 3 is a perspective view of an embodiment of an active electrode used in the machine according to the invention;

FIG. 4 is a plan view showing an application of the machine on a patient, wherein only the active electrodes located on different parts of the patient are shown; and

FIGS. 5a and 5b are views showing a section of a patient before and after the application of a visceral fat loss treatment by means of a machine based on the use of therapeutic molecular electromagnetic resonance according to the invention.

### DESCRIPTION OF A PREFERRED EMBODIMENT

In light of the aforementioned figures, and in accordance with the adopted numbering, one may observe therein a preferred exemplary embodiment of the invention, which comprises the parts and elements indicated and described in detail below.

Furthermore, the terms first, second, third and other similar terms in the description and in the claims are used to distinguish between similar elements and not necessarily to describe a sequential or chronological order. The terms may be interchanged in appropriate circumstances and embodiments of the invention may operate in sequences that are different from those described or illustrated herein.

Furthermore, the terms top, bottom, above, below, and other similar terms in the description and claims are used for descriptive purposes and not necessarily to describe relative positions.

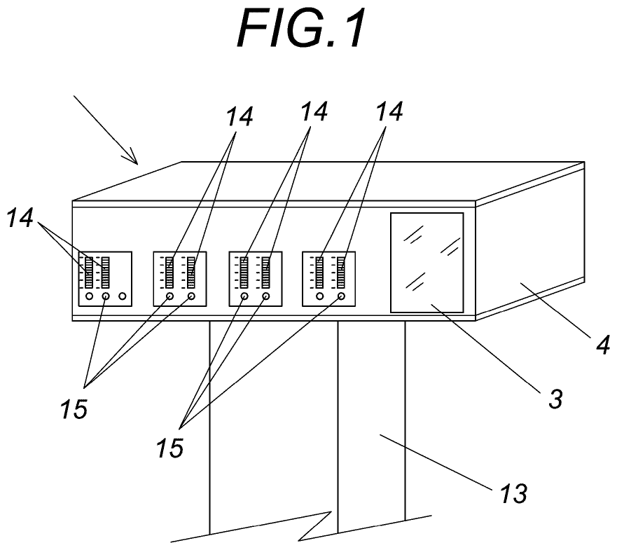

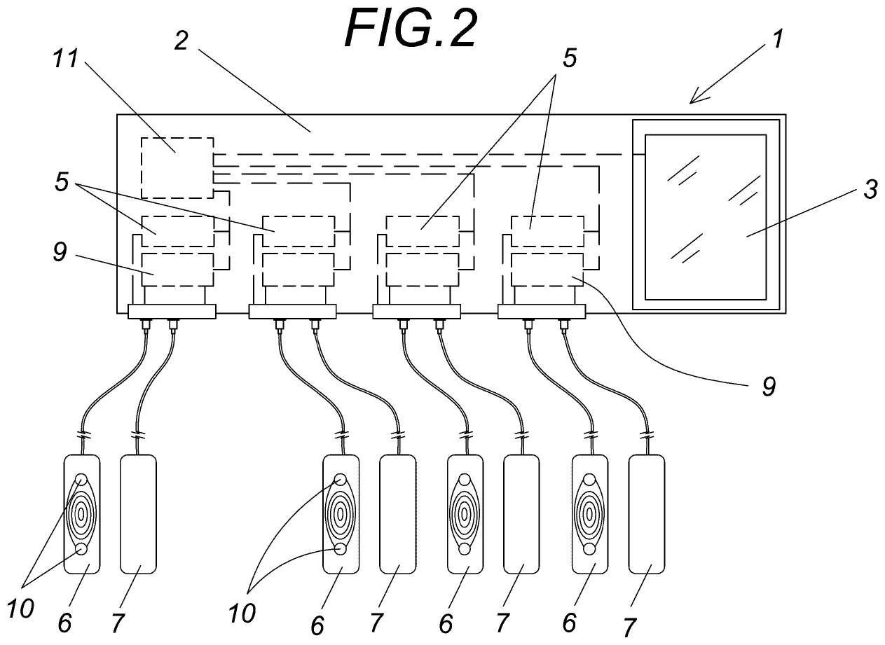

Thus, as can be seen in FIGS. 1 and 2, the machine, generally indicated by reference (1), is intended for treating body fat in different regions of the human body, as well as lipolysis, and is configured from a current-generating machine (2) located in a casing (4) comprising a touchscreen (3) provided for viewing and configuring the operating parameters. The casing (4) is supported by a pedestal (13) that includes a series of wheels on the lower base thereof that facilitate the movement of the machine (1).

Four channels are provided, each formed by a radiofrequency generator (5) to form radio waves, the signals generated by the radiofrequency generators being at a frequency between 0.3 MHz and 3 MHz. This radiofrequency generator (5) is linked to a temperature control circuit (9), active electrodes (6) that each provide 310 W of power, and monopolar passive or return electrodes (7) (meaning, each one of them is associated with an RF antenna) that are connected by means of connection ports (15).

For each of the electrodes to be coupled, the casing (4) also incorporates a graphic indicator (14) located on the same side wall where the screen (3) is located, which allows the frequency level applied in each one of the same to be easily viewed. The provision of an active electrode (6) and a passive electrode (7) for each of the operating channels allows a closed energy conduction circuit to be created, allowing the largest possible amount of energy to be delivered to the area of the body being treated.

The temperature control circuit (9) is connected to temperature sensors (10) incorporated in the monopolar active electrodes (6).

The current-generating machine (2) incorporates a computerised control module (11) associated with a computer program (software), connected to the patient interface that integrates the touchscreen (3), as well as to the radiofrequency generator (5) and to each channel, thus allowing, after the prior introduction of appropriate operating parameters for a specific treatment, the different treatments to be automated according to a specific pattern of times and temperatures that optimise the results, thereby allowing the evolution of the treatment to be managed and, in addition, to be able to develop all the phases thereof without the need for the intervention by any operator. In an alternative embodiment not shown, instead of a touchscreen, a plurality of pushbuttons can be used.

The provision of this computer program (software) allows the machine to work automatically for a preestablished period of time (for example, sixty minutes) depending on the parameters received by the active (6) and passive electrodes for superficial skin application, as well as the temperature sensors (10) and a series of physiological parameters previously entered into the computerised control module (11), such as age, weight, height, and abdominal circumference measurement.

In a functional alternative, the operator can order the machine to work manually, disconnecting the automated management applied by the software program through a pushbutton on the machine casing, such that the application of power applied to each of the active electrodes (6) is individually regulated by an operator, and thus the energy applied to each area of the body is chosen by the operator depending on the treatment.

The software program that is housed in the computerised control module (11) of the machine described herein allows a power of 1,240 watts to be delivered through the signals received by the various electrical and electronic components, thanks to the simultaneous action of the four channels during one treatment. In this way, in a treatment on the abdominal area of a patient, the software program manages the signals sent to each of the channels so that each of the active electrodes (6) delivers an energy value during a first predetermined period of time, generating an interference in order to multiply the effect of in-depth energy delivery. At this stage, the patient's skin temperature in the abdominal area is around a range of 40-55° C., the value of which is recorded by the energy-emitting elements, without causing burns in the area where the active electrodes are located due to the heat dissipation effect.

The computerised control module (11) executes the software program to apply the energy flow on the patient's skin for a predetermined time duration and to subsequently stop the energy flow transferred through at least one of the channels. Stopping of the flow of energy transferred through at least one of the channels is done automatically by executing the software program, although it can be stopped manually by means of a pushbutton on the machine.

Optionally, the machine (1) can incorporate a second brain-machine patient interface, consisting of an EEG capture terminal that detects the brain impulses of the patient or the operator and which in the example shown is configured by a headband-shaped fastening element, although it is possible to use other configurations, such as a cap or others, allowing said patient to influence the computerised control module (11) to which they are connected, when the temperature exceeds a certain value.

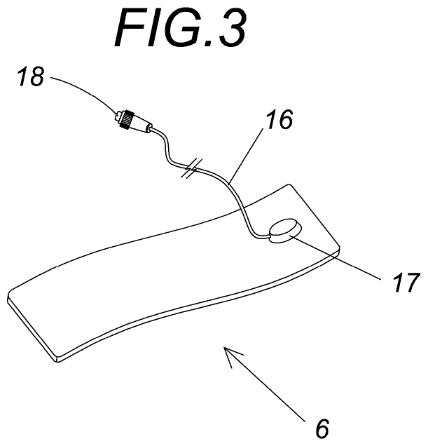

It must be mentioned that the active electrodes (6) and the passive electrodes have a flexible silicone coating that prevents the patient from suffering burns during a treatment with the machine described herein. To optimise energy transfer, an encapsulated gel coating is also provided, which improves adaptation to the entire surface of the patient's skin. FIG. 3 shows a preferred example of an active electrode (6) formed by an elongated flexible body of polymeric material that has a laminar shape, which is linked to a connector provided to transfer alternating current from the external power source, corresponding to the machine, to the active electrode (6). Each of the connector means is essentially made up of a cable (16) with a variable length that protrudes from a protuberance (17) on one of the faces of the laminar plate (1), and a specific connection socket (18).

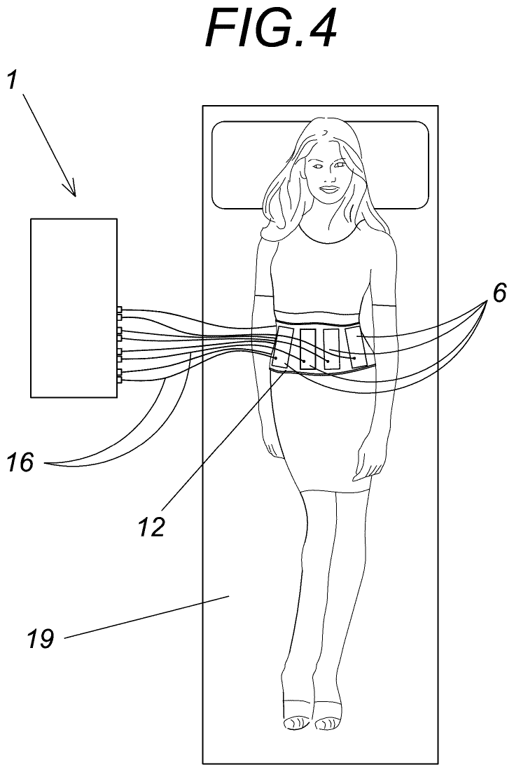

FIG. 4 shows an application of the machine on a patient who is in a stretched position on a conventional stretcher (19), wherein only the active electrodes (6) located on different parts of the patient are shown. The passive electrodes located on the back of the patient have not been represented since they would not be visible, as they are located on the patient's lumbar area. In this way, the arrangement is such that it allows for the electromagnetic energy flow to cross, generating a total of 1,240 W from the sum of all active electrodes (6) in the area where the reduction of visceral fat is carried out, allowing for a reduction of visceral fat without any negative effects on the patient. In other words, the flow of energy applied by the active electrodes (6) is focused on a specific region where there is an interest in reducing the volume of visceral fat, the area of application of the electrodes being detected by energy absorption.

As can be seen, the active and passive electrodes that make up part of the machine are grouped or coupled in a belt (12) that can be adjusted in size to adapt to the contour of each patient, through conventional fastening means, such as Velcro® straps, for example. This belt-like accessory incorporates the radiofrequency-emitting and—receiving elements within the structure thereof, eliminating the need to apply individual antennas to transfer the radiofrequency.

Treatment in a Patient

An adult patient underwent a visceral fat reduction treatment using the previously described machine for 10 sessions and over a period of 12 days, each session lasting between 50 minutes and 120 minutes. Table 1 shows the total energy applied through each of the active electrodes (6) of the four channels in sessions 1, 5 and 10, as well as the average temperature value obtained from the temperature sensor in the four active electrodes (6).

TABLE 1

| Data per session | Session 1 | Session 5 | Session 10 |
|---|---|---|---|
| Total kilojoules | 192 | 267 | 232 |
| Average temperature in ° C. | 44 | 47 | 45 |

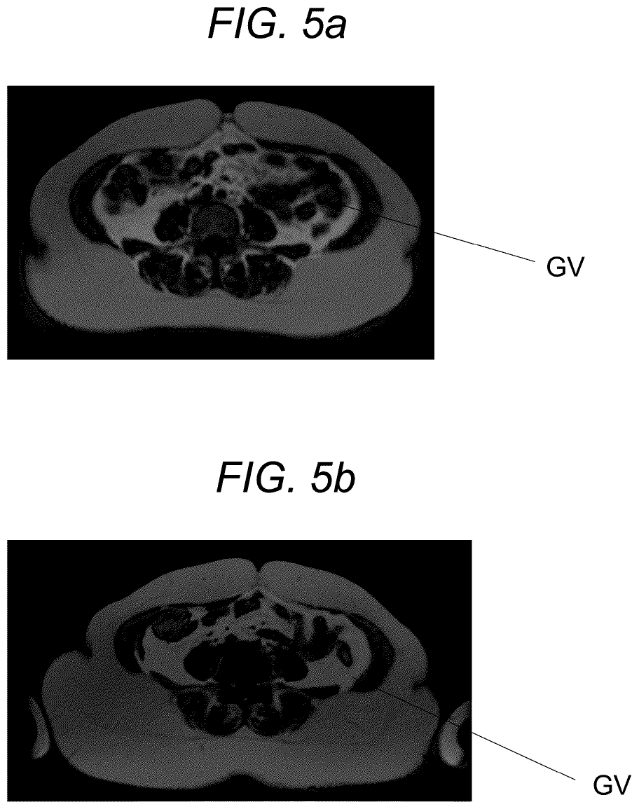

The results obtained from the treatment on the patient by means of the machine are presented below in table 2 and FIGS. 5a and 5b, in the first and tenth treatment sessions.

FIG. 5a shows an image of a magnetic resonance before applying treatment, whereas FIG. 5b shows a resonance after finishing the 10-session treatment, and thus it can be seen how the volume of visceral fat corresponding to the central part of FIGS. 5a and 5b has changed, the volume of visceral fat, indicated by the reference (VF), being smaller in

TABLE 2

| | Session | Session | Variation |
|---|---|---|---|
| patient | 1 | 10 | in % |
| Visceral fat (in grams) | 1515.0 | 955.0 | −36.96% |
| Subcutaneous fat (in grams) | 7563.0 | 6788.0 | −10.25% |
| Visceral fat (in cm³) | 1683.0 | 1061.0 | −36.96% |
| Subcutaneous fat (in cm³) | 7542.0 | 8403.0 | 11.42% |

The results in table 2 show a significant weight loss of visceral and subcutaneous fat after the application of the ten sessions and, consequently, a noteworthy reduction in the patient's weight, resulting in a significantly thinner silhouette, the patient experiencing a reduction of 16 cm in the length of the contour of their waist area and losing 4 kg of weight. This type of body modification has not been achieved or scientifically demonstrated with magnetic resonance diagnostic systems until now, a fact which represents a major advance in the state of the art. This case is circumscribed in a scientific study carried out with twenty subjects, in which all experienced a statistically significant reduction in their visceral fat in a short period of time, in this case twelve days, and without being subjected to diet or exercise. For these reasons, this technology represents an innovation in the state of the art that makes it possible to provide a solution to a public health problem with no other current solution available beyond diet and exercise.

The details, shapes, dimensions and other accessory elements used to manufacture the machine of the invention may be suitably substituted for others which do not depart from the scope defined by the claims included below.

1. A machine for treating visceral fat, the machine comprising:
a current-generating machine, first patient interface means, a computerised control module connected to the patient interface, said computerised control module module being capable of automating treatments according to a pattern of times and temperatures, after the necessary parameters are entered,
wherein it comprises four channels, each of them provided with a radiofrequency generator connected to active electrodes, and passive electrodes, a temperature control circuit connected temperature sensors arranged in the active electrodes, and a specific software program housed in the computerised control module that provides a power value for each of the channels in an asynchronous way, such that it simultaneously readjusts the power value of each of the four channels, depending on at least the energy flow value emitted by the active electrodes of each channel and the energy flow value received from each of the passive electrodes of the corresponding channels.

2. The machine according to claim 1, wherein the first interface means comprise a touchscreen.

3. The machine according to claim 1, wherein the signals generated by the radiofrequency generators of each of the channels are wave signals at a frequency between 0.3 MHz and 3 MHz.

4. The machine according to claim 1, wherein the active electrodes and the passive electrodes are the type that are applied to the skin, as they are provided with a surface contact region intended to make contact with the patient's skin.

5. The machine according to claim 1, wherein the active electrodes and the passive electrodes are located on a belt intended to be placed around a patient.

6. The machine according to claim 1, wherein the computerised control module executes the software program to apply the energy flow on the patient's skin for a predetermined time period and to subsequently stop the flow of energy transferred through at least one of the channels.

7. The machine according to claim 6, wherein the computerised control module executes the software program to automatically regulate and/or stop the flow of energy transferred through at least one of the channels.

8. The machine according to according to claim 1, wherein each of the outputs of the active electrodes has a maximum power of 310 W.

9. The machine according to according to claim 1, wherein the total output of the four channels has a maximum power of 1240 W.

10. The machine according to claim 1, wherein the active electrodes are grouped on a strap envisaged to be placed on the surface of the patient's body.

11. The machine according to claim 1, wherein the passive electrodes (7) are grouped on a strap envisaged to be placed on the surface of the patient's body.

12. A method for personalised treatment of visceral fat using a machine, for treating visceral fat,
the machine comprising:
a current-generating machine, first patient interface means, a computerised control module connected to the patient interface, said computerised control module being capable of automating treatments according to a pattern of times and temperatures, after the necessary parameters are entered,
wherein it comprises four channels, each of them provided with a radiofrequency generator connected to active electrodes, and passive electrodes, a temperature control circuit connected temperature sensors arranged in the active electrodes, and a specific software program housed in the computerised control module that provides a power value for each of the channels in an asynchronous way, such that it simultaneously readjusts the power value of each of the four channels, depending on at least the energy flow value emitted by the active electrodes of each channel and the energy flow value received from each of the passive electrodes of the corresponding channels;
the method comprising:
the application of an energy flow through the arrangement on the patient's skin, a plurality of active electrodes for the input of an energy flow into the patient's body and passive electrodes for the output of the energy flow on the patient's skin, each active electrode and passive electrode being linked to a corresponding channel, and
wherein by means of a software program each of the channels has the ability to share data with each other and apply an energy flow based on the ratio of the input energy flow value to the output energy flow value and the amount of body fat present in the patient's body area, located between the active electrodes and the passive electrodes.

13. The method for personalised treatment of visceral fat according to claim 12, wherein the software program is executed to apply an energy flow with a specific temperature value based on the ratio between the input energy flow value and the output energy flow value.

14. The method for personalised treatment of visceral fat according to claim 12, wherein the active electrodes and passive electrodes have a radial arrangement around the patient, in such a way that active electrodes face the passive electrodes associated with one same energy flow channel.
